(19) United States
(12) Patent Application Publication
(10) Pub. No.: US 2012/0107922 A1
(43) Pub. Date: May 3, 2012

(54) TAMPERING DEVICE USED DURING EMBEDDING OF BIOLOGICAL TISSUE SAMPLES

(76) Inventors: Nico Ooms, Weatherford, TX (US); Laster Williams, Weatherford, TX (US)

(21) Appl. No.: 13/285,902

(22) Filed: Oct. 31, 2011

Related U.S. Application Data
(60) Provisional application No. 61/409,640, filed on Nov. 3, 2010.

(51) Int. Cl. C12M 1/26

(52) U.S. Cl. 435/309.1

(57) ABSTRACT

A tampering device may comprise a tamper, an elongated handle, and an insulator rod. The elongated handle may have a first end and a second end. The insulator rod may be adapted to connect to the tamper and the first end of the elongated handle. The elongated handle may have a gripping ridge and a plurality of valleys. The valleys may include a plurality of dimples to increase the surface of interface between the user's fingers and the handle.

### RELATED APPLICATIONS

The present application claims benefit of U.S. Provisional Application No. 61/409640 filed on Nov. 3, 2010, the entirety of which is incorporated herein by reference.

### BACKGROUND OF THE INVENTION

The present invention generally relates to a tampering device, and more specifically relates to a tampering device used for embedding of biological tissue samples.

Current tampering devices that are used in the histology profession require the use of forceps as shown in FIG. 2-3 or small tampering devices as shown in FIG. 4. In the case of forceps, the user may grasp a T shaped tamper with forceps and may need to apply a significant force to hold on to the T shaped tamper. The user may apply a down force onto the T shaped tamper. Small contact areas between the T shaped tamper and forceps may easily result in rocking motion while using it.

The small tampering devices may require squeezing the end of the shaft while applying a down force. Both these devices cause squeezing the end of the shaft while applying a down force. In addition, these devices may cause straining, muscle fatigue and discomfort to the hand when used for extended periods of time. Pressure that is applied to tissue while embedding is a critical factor. The pressure will ensure that the tissue is flat while the liquid paraffin hardens.

Therefore, it can be seen that there is a need for an ergonomic tampering device used during embedding of biological tissue samples.

### SUMMARY OF THE INVENTION

In one aspect of the present invention, a tampering device comprises a tamper; an elongated handle having a first end and a second end; and an insulator rod adapted to connect to the tamper and the first end of the elongated handle.

In another aspect of the present invention, a tampering device comprises a tamper having a bottom, a top, and a plurality of sidewalls integrally attached to and surround peripheries of the bottom and the top; and an elongated handle operationally connected to the tamper.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is a perspective view of typical devices used in embedding of biological tissue samples;

FIG. 3 is a perspective view of another typical device used in embedding of biological tissue samples; and;

FIG. 4 is a perspective view of small tampering devices used in embedding of biological tissue samples.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an exemplary embodiment of the present invention generally provides a tampering device used for embedding of biological tissue samples.

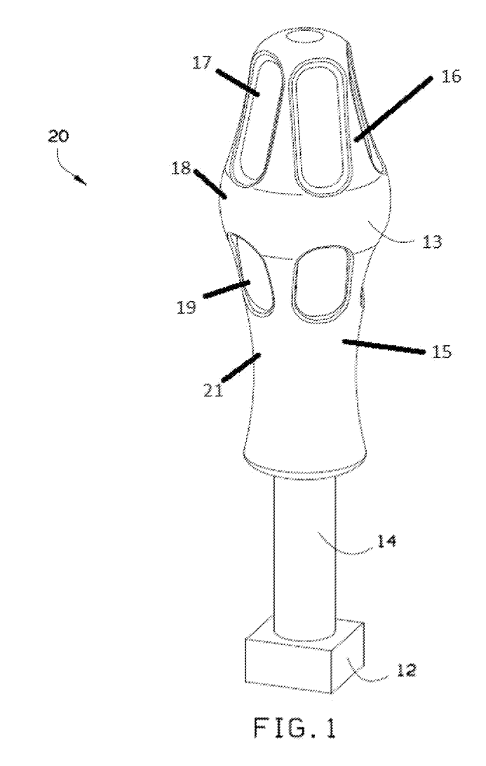

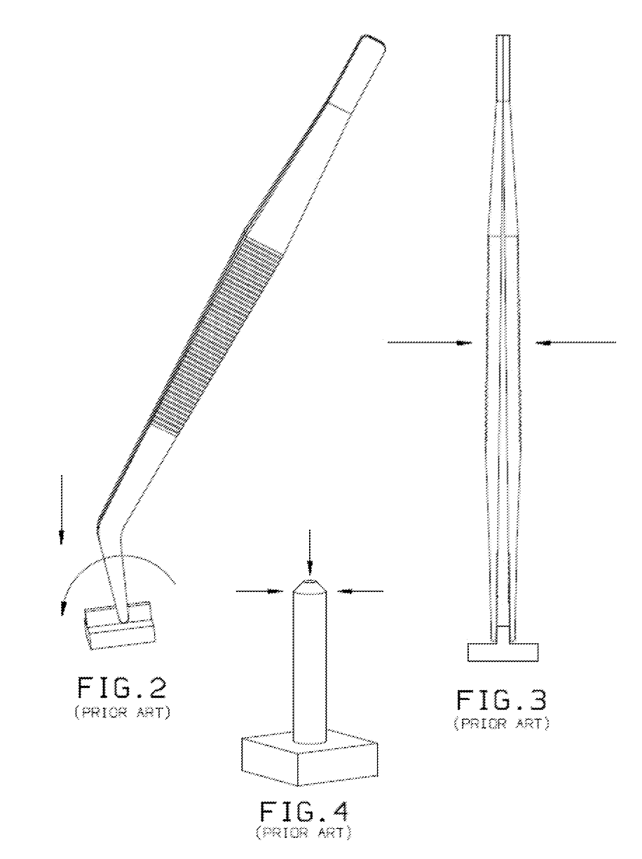

As shown in FIG. 1, in an exemplary embodiment of the present invention, the tampering device 20 may be configured and designed to assist in the preparation of biological tissue samples in a histology lab. It may have an ergonomic handle 13, which is adapted to be grasped by a hand. The handle 13 may have undulating surfaces 21 to provide a comfortable, non-slip interface with the user's hand. The handle 13 may have a gripping ridge 18 with gripping valleys 15 and 16. The valleys 15 and 16 may include a plurality of ovoid dimples 19 and 17, respectively, to increase the surface of interface between the user's fingers and the handle 13. The handle 13 may have other shapes and sizes.

The handle 13 may be designed to avoid strains caused to the hand or fingers of the user while the user applies force to it. The handle 13 should be able to resist harsh chemicals that are used to clean and disinfect materials and devices used in the biological or chemical lab, such as, histology labs.

In an exemplary embodiment of the present invention, the insulator rod 14 may be made of materials that have low thermal conductive characteristics. The materials used may have proper mechanical properties which may not bend or brake when pressure is applied. The insulator rod 14 may be made of hard supportive materials that meet thermal properties and mechanical properties known in the art, such as plastics, ceramics, for example. The width and depth of the insulator rod 14 may not be larger than the tamper 12 to eliminate any instability during the use of the device 20.

In an exemplary embodiment, the tamper 12 may be made of metal that is good heat conductor and compatible with materials used in a histology lab. The tamper 12 may be electrochemically treated to provide a smooth surface finish which may reduce the adhesion of potential biological tissue sample or any other embedding compound used.

A technician may apply high pressure force onto the sample without having to apply a great force. The tamper 12 may have a size larger than two inches wide by two inches long, for example. In an exemplary embodiment, the tamper 12 may be symmetrical for ease of operation.

The insulator rod 14 may be mounted in the center of the tamper 12. The bottom surface of the tamper 12 may be flat. No protrusion or recession of mounting hardware may be allowed at the bottom surface of the tamper 12. Mounting the insulator rod 14 in the center of the tamper 12 may allow the user to apply high pressure on the biological tissue and embedding material with minimal force.

The ergonomic handle 13 may be mounted in the center of the insulator rod 14, which may allow users to apply high pressure with minimal force. The use of the tampering device may apply a rotational motion while applying pressure on the sample. In an exemplary embodiment, the ergonomic handle 13 may be permanently attached to the insulator rod 14 to prevent separation from the insulator.

To make the present invention, a user may obtain the relevant parts, which include the tamper 12, the insulator rod 14, and the handle 13. The user may connect the parts by screws or adhesives to put the parts together.

In an exemplary embodiment, the length of the insulator rod 14 may vary. Making the insulator rod 14 short may limit the view that the histology technician has over the sample and may not be recommended. Lengthening the insulator 14 may result in a design that is unstable while applying force onto the sample that needs to be prepared.

An exemplary embodiment of the present invention may provide an easy operation while embedding biological tissue samples into liquid paraffin. An exemplary embodiment of the present invention may allow users to apply great pressure onto bones or sections of bones by placing a thumb on the top of the handle and placing a force onto the ergonomic handle 13.

It should be understood, of course, that the foregoing relates to exemplary embodiments of the invention and that modifications may be made without departing from the spirit and scope of the invention as set forth in the following claims.

We claim:

1. A tampering device, comprising:
a tamper;
an elongated handle having a first end and a second end; and
an insulator rod adapted to connect to the tamper and the first end of the elongated handle.

2. The tampering device of claim 1, wherein the tamper comprises a bottom, a top, and a plurality of sidewalls integrally attached to and surround peripheries of the bottom and the top.

3. The tampering device of claim 1, wherein the tamper has a flat bottom.

4. The tampering device of claim 1, wherein the tamper is electrochemically treated so that the tamper can resist harsh conditions.

5. The tampering device of claim 1, wherein the insulator is mounted at a center of the tamper.

6. The tampering device of claim 1, wherein the tamper is symmetrical around the center of the tamper.

7. A tampering device comprising:
a tamper having a bottom, a top, and a plurality of sidewalls integrally attached to and surround peripheries of the bottom and the top; and
an elongated handle operationally connected to the tamper.

8. The tampering device of claim 7, wherein the elongated handle has a gripping ridge.

9. The tampering device of claim 7, further comprising a plurality of valleys.

10. The tampering device of claim 7, further comprising a plurality of dimples to increase surface of interface between fingers and the handle.
